(19) United States
(12) Patent Application Publication
(10) Pub. No.: US 2026/0177843 A1
(43) Pub. Date: Jun. 25, 2026

(54) APPARATUS AND METHOD FOR CONTROLLING FOCUS THROUGH PROGRESSIVE LEARNING

(71) Applicant: ELECTRONICS AND TELECOMMUNICATIONS RESEARCH INSTITUTE, Daejeon (KR)

(72) Inventors: Moo Seop KIM, Daejeon (KR); Young Mi Song, Daejeon (KR); Sungjun Wang, Daejeon (KR); Chi Yoon Jeong, Daejeon (KR); SuGil Choi, Daejeon (KR)

(21) Appl. No.: 19/327,288

(22) Filed: Sep. 12, 2025

(30) Foreign Application Priority Data
Dec. 24, 2024 (KR) 10-2024-0195940

(51) Int. Cl. G02C 7/08; G06F 3/01; G06T 7/50; G06T 7/70

(52) U.S. Cl. CPC G02C 7/081; G06F 3/015; G06T 7/50; G06T 7/70; G06T 2207/20081; G06T 2207/30201

(57) ABSTRACT

The present disclosure relates to a visual assistance device that controls focus through progressive learning. The device includes: a data acquisition unit configured to collect visual information of an object gazed at by a user, based on the user's biometric, visual, and auxiliary signals; a gaze analysis unit configured to analyze the user's gaze using the visual information and to generate focus control information through a trained focus prediction model; a control unit configured to produce a control signal to adjust the focus of the device based on the focus control information and to receive user feedback on the set focus; and a data learning unit configured to acquire training data for the focus prediction model, detect errors in the focus control information, and update the model by retraining it with the revised data.

### BACKGROUND

### 1. Technical Field

The present disclosure relates to a method for controlling focus, and more particularly, to an apparatus and method for controlling focus through progressive learning.

### 2. Related Art

In general, visual assistance devices, such as smart glasses or wearable healthcare devices, are equipped with a sensor module mounted on the temple of the glasses for collecting a user's biometric signals. For example, products such as Google Glass and Vuzix smart glasses have been developed, some of which provide functions for monitoring biometric signals.

However, conventional visual assistance devices have difficulties in properly responding to changes in a user's vision or age-related deterioration. In particular, many existing focus control methods provide only a fixed focal distance or rely on manual adjustment, causing inconvenience for users who need to view objects at varying distances. Accordingly, there is a need for a new method capable of automatically adjusting focus by tracking and analyzing a user's gaze information and changes in vision in real time.

Korean Unexamined Patent Publication No. 10-2021-0124870 discloses a selective weight update method for reducing latency and memory consumption in a progressive learning model.

However, Korean Unexamined Patent Publication No. 10-2021-0124870 does not disclose a method for automatically adjusting focus through learning that tracks and analyzes a user's gaze information and changes in vision in real time.

Accordingly, there is a need for research on methods capable of automatically adjusting focus through learning that tracks and analyzes a user's gaze information and changes in vision in real time.

### PRIOR ART DOCUMENTS

(Patent Document 1) Korean Registered Patent No. 10-2527972

### SUMMARY

Accordingly, the present disclosure is directed to providing, as a first objective, a method for real-time focus control tailored to a user's gaze. Specifically, the present disclosure provides a method in which the position and distance of an object being gazed at by the user are tracked and analyzed in real time, and the focus of a variable-focus lens is automatically adjusted, allowing the user to maintain consistent visual clarity across various distances and environments. For example, when a user transitions from reading a book to viewing a television located farther away, the present disclosure immediately detects the user's gaze shift and automatically adjusts the lens focus from near to far distance, thereby reducing visual fatigue and providing a more comfortable viewing experience. Such a real-time focus control method can also be extremely useful for checking road signs while driving or for tracking rapidly moving objects during sports activities.

Additionally, the present disclosure is directed to providing, as a second objective, a method for implementing a continuous learning model for personalized vision correction. Specifically, the present disclosure proposes constructing a customized focus prediction model based on a user's vision condition (e.g., myopia, hyperopia, or astigmatism) and gaze patterns, and continuously learning and improving the model to provide focus adjustment optimized for the user's vision changes and visual requirements. For example, when a user frequently performs computer work that involves close-range gazing, the present disclosure analyzes the user's gaze patterns and feedback data to automatically learn and adjust focus settings optimized for the user's working environment. Through this approach, the user can conveniently continue working without the need to manually remove or adjust glasses as the focus is automatically maintained.

Further, the present disclosure is directed to providing, as a third objective, a method for adaptive focus control that responds to environmental changes. Specifically, the present disclosure provides a method for adaptively adjusting focus based on environmental changes (e.g., indoor, outdoor, lighting conditions) and visual situations encountered by the user. For example, when a user reads a book in a dimly lit environment or gazes at a sign outdoors, the present disclosure provides a method of automatically adjusting the focus settings according to each situation. When the user operates a smartphone in a dark indoor environment, the present disclosure adjusts the focus by considering not only the user's gaze position but also the lighting conditions, thereby minimizing visual fatigue and providing a clearer view. Conversely, when the user gazes at a distant sign in a brightly lit outdoor environment, the focus is automatically adjusted to a far distance.

Moreover, the present disclosure is directed to providing, as a fourth objective, a method for maintaining and improving the learning performance of a focus prediction model through progressive learning. Specifically, the present disclosure continuously collects a user's real-time response data through a progressive learning algorithm and utilizes the collected data as training data for the focus prediction model, thereby enabling continuous improvement of the model's performance. Through this approach, the system can autonomously adapt to factors such as changes in the user's vision, fatigue level, and concentration. For example, when a user experiences visual fatigue after prolonged computer use, the present disclosure can detect such a condition and provide methods such as recommending visual rest or adjusting the speed of focus control. Furthermore, if the user's vision deteriorates due to presbyopia, the focus prediction model can be retrained according to the new vision state to maintain visual comfort.

In addition, the present disclosure is directed to providing, as a fifth objective, a method for precise focus prediction through the integrated analysis of various biometric and visual signals. Specifically, the present disclosure analyzes biometric signals, such as electrooculography (EOG) and electroencephalography (EEG), together with visual signals, in an integrated manner to evaluate a user's gaze movement, concentration level, and fatigue, and provides a method for precisely adjusting the focus of a variable-focus lens based on such evaluations. For example, when a user begins to experience fatigue while concentrating on a screen, the present disclosure can analyze changes in EEG signals and the stability of EOG signals to either preemptively adjust the focus or provide visual fatigue mitigation measures before the user blinks or turns their head.

Furthermore, the present disclosure is directed to providing, as a sixth objective, an intelligent focus control method that responds to long-term changes in a user's vision. Specifically, the present disclosure provides a method for continuously updating and improving a focus prediction model to accommodate long-term vision changes caused by aging or diseases.

Through this approach, users can continuously maintain a comfortable visual experience. For example, when a user develops presbyopia or progresses toward cataracts, the present disclosure enables real-time detection of such conditions and continuously accumulates corresponding training data to improve the model. As a result, the user can automatically receive optimal focus adjustments without needing to replace the lens, even as vision changes occur.

The technical problems to be addressed by the present invention are not limited to those described above. Other technical problems that are not explicitly mentioned herein will be clearly understood by those of ordinary skill in the art based on the following descriptions.

The present disclosure provides a visual assistance device for controlling focus through progressive learning, comprising: a data acquisition unit configured to collect visual information of an object being gazed at by a user based on biometric signals, visual signals, and auxiliary signals of the user; a gaze analysis unit configured to analyze gaze information related to the user's gaze using the collected object visual information and to generate focus control information for adjusting the focus of the visual assistance device through a trained focus prediction model; a control unit configured to generate control signals for adjusting the focus of the visual assistance device based on the focus control information, and to receive feedback information regarding the user's response to the adjusted focus; and a data learning unit configured to acquire training data for the focus prediction model, monitor whether errors occur in the focus control information, and retrain the focus prediction model using updated training data to improve the model.

In addition, the present disclosure is characterized in that the visual information of the object includes information regarding the position, distance, and direction of the object currently being gazed at by the user in an image.

In addition, the present disclosure is characterized in that the gaze information includes information regarding the user's gaze position, the gaze movement path, and the fixation time on the object being gazed at.

In addition, the present disclosure is characterized in that the gaze analysis unit classifies the user's gaze patterns based on the gaze movement path and the fixation time information, and generates focus control information corresponding to each gaze pattern.

In addition, the present disclosure is characterized in that the gaze analysis unit determines whether the set focus matches based on the feedback information.

In addition, the present disclosure is characterized in that, when the set focus does not match, the data learning unit adds the feedback information to the training data and updates the training data.

In addition, the present disclosure is characterized in that the data learning unit monitors whether an error has occurred in the focus control information by comparing the frequency of focus prediction failures with a threshold value.

In addition, the present disclosure is characterized in that, when the frequency of focus prediction failures is greater than or equal to the threshold value, the data learning unit retrains the focus prediction model.

In addition, the present disclosure is characterized in that the data learning unit trains the focus prediction model through an initial training phase, an adaptive training phase, and an operational training phase.

In addition, the present disclosure is characterized in that the biometric signals include electrooculography (EOG) signals, electroencephalography (EEG) signals, heart rate, and skin conductance; the visual signals include image information for monitoring the user's eyes and gaze and information regarding the distance to an object; and the auxiliary signals include movement information and brightness information of the user, measured by a plurality of sensors.

In addition, the present disclosure is characterized in that a method for controlling focus through progressive learning comprises: collecting visual information of an object being gazed at by a user based on the user's biometric signals, visual signals, and auxiliary signals; analyzing gaze information related to the user's gaze using the collected object visual information and generating focus control information for adjusting the focus of the visual assistance device through a trained focus prediction model; generating a control signal for adjusting the focus of the visual assistance device based on the focus control information and receiving feedback information regarding the user's response to the set focus; and monitoring whether an error has occurred in the focus control information and updating the focus prediction model by retraining it using updated training data obtained through the feedback.

In addition, the present disclosure is characterized in that the step of generating the focus control information includes: classifying the user's gaze patterns based on the gaze movement path and the fixation time information; and generating focus control information corresponding to each classified gaze pattern.

In addition, the present disclosure is characterized in that the step of generating the focus control information includes a step of determining whether the set focus matches based on the feedback information.

In addition, the present disclosure is characterized in that the method further includes a step of adding the feedback information to the training data and updating the training data when the set focus does not match.

In addition, the present disclosure is characterized in that the step of monitoring whether an error has occurred in the focus control information includes a step of comparing the frequency of focus prediction failures with a threshold value.

In addition, the present disclosure provides an AI device for controlling focus through progressive learning, comprising:
a communication unit configured to receive visual information of an object being gazed at by a user based on the user's biometric signals, visual signals, and auxiliary signals;
a memory configured to store a focus prediction model; and an AI processor functionally connected to the communication unit and the memory, configured to control overall operations of the AI device, wherein the AI processor is configured to analyze gaze information related to the user's gaze using the visual information of the object, generate focus control information for adjusting the focus of a visual assistance device through the trained focus prediction model, generate a control signal for adjusting the focus of the visual assistance device based on the focus control information, receive feedback information regarding the user's response to the set focus, acquire training data for training the focus prediction model, monitor whether an error has occurred in the focus control information, and update the focus prediction model by retraining it using updated training data.

The present disclosure enables the real-time collection and analysis of a user's gaze and response information through biometric signals (e.g., EOG, EEG) and visual signals, thereby continuously training and improving a focus prediction model tailored to the individual characteristics of each user. As a result, the present disclosure can perform much more precise and personalized focus control compared to conventional simple gaze tracking methods. In particular, the present disclosure is distinguished from conventional technologies by adapting the focus prediction model to various situations such as changes in the user's vision, aging, and changes in the usage environment. Unlike conventional technologies that rely on a fixed model, the present disclosure provides the advantage of being able to immediately respond to the user's changing visual needs by updating the model through adaptive learning stages.

In addition, the present disclosure can maintain and improve the learning performance of the focus prediction model through continuous and progressive learning. Moreover, the improvement of the model through progressive learning can contribute to continuously enhancing prediction accuracy and optimizing the user experience by utilizing individual user response information as training data.

Furthermore, the present disclosure allows for the implementation of a flexible focus prediction model capable of adapting to various environmental changes.

Furthermore, the present disclosure provides the effect of significantly improving the user's satisfaction with the visual experience by enhancing the accuracy of focus prediction through the integration of various sensor data. In particular, the present disclosure enables immediate focus adjustment based on user responses by analyzing various biometric signals and visual data in real time, thereby providing a natural visual experience to the user.

The effects obtainable from the present invention are not limited to those described above, and other effects not explicitly mentioned herein will be clearly understood by those of ordinary skill in the art based on the following descriptions.

The accompanying drawings, which are included as part of the detailed description to assist in the understanding of the present invention, illustrate embodiments of the invention and, together with the detailed description, serve to explain the technical features of the invention.

### BRIEF DESCRIPTION OF THE DRAWINGS

FIG. 1 is a block diagram illustrating an example of an internal configuration of a visual assistance device proposed in the present disclosure.

FIG. 2 is a diagram illustrating an example of an operation method of a visual assistance device through progressive learning proposed in the present disclosure.

FIG. 3 is a detailed block diagram illustrating an example of the internal configuration of the visual assistance device proposed in the present disclosure.

FIG. 4 is a block diagram illustrating an example of an AI device to which the method proposed in the present disclosure can be applied.

FIG. 5 is a flowchart illustrating an example of a user-customized focus control method through a progressive learning method proposed in the present disclosure.

FIG. 6 is a block diagram illustrating a computer system for implementing a method according to an example embodiment of the present disclosure.

### DETAILED DESCRIPTION OF THE EMBODIMENTS

It should be noted that the technical terms used in the present disclosure are employed merely for the purpose of describing specific embodiments and are not intended to limit the spirit of the technology disclosed herein. Unless otherwise defined, the technical terms used in the present disclosure should be interpreted as having meanings generally understood by those of ordinary skill in the art to which the present disclosure pertains, and should not be interpreted in an unduly broad or unduly narrow sense. In cases where the technical terms used herein fail to accurately express the spirit of the technology disclosed, such terms should be replaced with appropriate terms that can be correctly understood by those of ordinary skill in the art. In addition, general terms used in the present disclosure should be interpreted according to definitions provided in dictionaries or based on the context in which they are used, and should not be construed in an excessively narrow sense.

Terms including ordinals such as “first” and “second” used in the present disclosure may be employed to describe various elements, but such elements should not be limited by these terms. These terms are only used to distinguish one element from another. For example, without departing from the scope of the present invention, a first element may be referred to as a second element, and similarly, a second element may be referred to as a first element.

Hereinafter, exemplary embodiments disclosed in the present disclosure will be described in detail with reference to the accompanying drawings. Throughout the drawings, the same reference numerals are used to designate the same or similar components, regardless of the figure number, and redundant descriptions thereof will be omitted.

In describing the technology disclosed in the present disclosure, detailed descriptions of related known technologies will be omitted when it is determined that such descriptions could obscure the essence of the disclosed technology. In addition, it should be noted that the accompanying drawings are provided merely to facilitate the understanding of the spirit of the disclosed technology and should not be construed as limiting the scope of the invention.

Before specifically examining the user-customized focus control method through progressive learning proposed in the present disclosure, the technical limitations of existing visual assistance devices will be briefly reviewed.

First, conventional visual assistance devices are limited by fixed-focus mechanisms. Most conventional visual assistance devices (e.g., eyeglasses, lenses) provide a fixed focus for a specific distance. For example, reading glasses designed for close-range tasks or standard eyeglasses assisting distant vision do not allow focus adjustment when the user shifts their gaze. As a result, users are inconvenienced by having to remove or switch glasses when alternating between viewing distant and nearby objects. This fixed-focus mechanism becomes particularly problematic in cases of age-related vision deterioration, such as presbyopia. For instance, users over the age of 40 may require reading glasses to assist with near vision but must remove them again for distant vision, causing additional inconvenience.

Second, conventional visual assistance devices are inconvenient to use due to manual focus adjustment mechanisms. Some conventional visual assistance devices provide methods for manually adjusting the focus; however, these require active operation by the user. For example, focus-adjustable glasses may require the user to press a button attached to the device to achieve the desired focus. Although such methods enable precise adjustment, they are inconvenient and time-consuming because the user must manually adjust the focus each time it is needed. Accordingly, manual adjustment mechanisms are not suitable for environments that require rapid focus changes. For instance, during a sporting event or when observing moving objects, users may miss important moments or information due to the limitations of manual operation.

Third, conventional visual assistance devices have difficulty adapting to changes in a user's vision over time. When a user's vision changes over time, conventional visual assistance devices or focus-adjustable lenses have the disadvantage of being unable to effectively respond to such changes. For example, in situations where vision rapidly deteriorates due to aging or medical conditions, conventional fixed-focus glasses or variable-focus lenses become inefficient. Although some automatic focus adjustment devices have been developed, they still fail to reflect the user's individual gaze patterns or real-time vision changes. For instance, methods that merely calculate the distance between the user and an object to adjust focus cannot account for individual visual characteristics, fatigue, or changes in concentration, thereby causing user discomfort.

Accordingly, in order to overcome the limitations of conventional visual assistance devices described above, the present disclosure proposes a method and a visual assistance device capable of real-time gaze tracking and focus control, enabling user-customized vision correction, and continuously improving the performance of a focus prediction model through progressive learning.

Hereinafter, the user-customized focus control method through progressive learning and the visual assistance device supporting the same, as proposed in the present disclosure, will be described in detail with reference to the accompanying drawings.

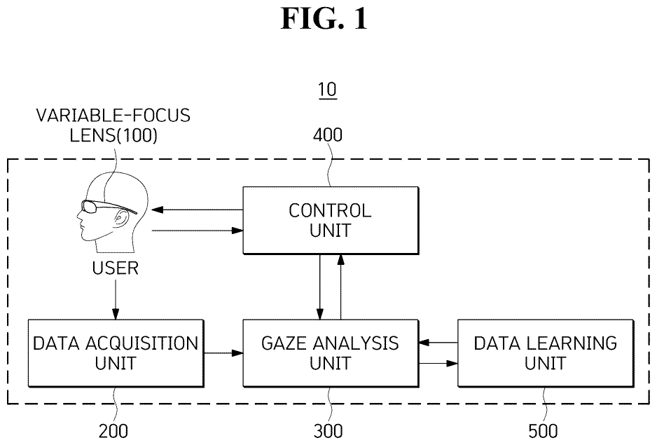

FIG. 1 is a block diagram illustrating an example of an internal configuration of the visual assistance device proposed in the present disclosure.

Referring to FIG. 1, the visual assistance device 10 may include a variable-focus lens 100, a data acquisition unit 200, a gaze analysis unit 300, a control unit 400, and a data learning unit 500.

Alternatively, the visual assistance device may refer to the variable-focus lens itself. In this case, the visual assistance device may include the data acquisition unit, the gaze analysis unit, the control unit, and the data learning unit, and the terms “visual assistance device” and “variable-focus lens” may be used interchangeably.

The visual assistance device proposed in the present disclosure tracks and analyzes the user's gaze information in real time, and adjusts the focus based on the position and distance of the object being gazed at, thereby providing optimal visual information to the user. In addition, the visual assistance device controls the variable-focus lens through continuous and progressive learning in response to changes in the user's vision or aging.

Referring to FIG. 1, the variable-focus lens 100 may generally refer to eyeglasses worn by the user or a device implemented within such eyeglasses.

The data acquisition unit 200 is a component that collects visual information input through the visual assistance device. It collects information regarding the position, distance, and direction of the object (or target) currently being gazed at by the user, based on visual information such as images or muscle signals around the eyes.

The gaze analysis unit 300 uses the information collected by the data acquisition unit to predict the object being gazed at by the user, generates information required for adjusting the focus of the variable-focus lens in the visual assistance device worn by the user, and transmits the generated focus control information to the control unit.

In addition, the gaze analysis unit 300 receives user response information (i.e., feedback) in response to the focus control information transmitted to the control unit, and determines whether the predicted focus from the focus prediction model matches. If the feedback information does not match the focus predicted by the visual assistance device, the visual assistance device adds the received user response information as new training data.

The control unit 400 is a component that controls the overall operation of the visual assistance device and controls the variable-focus lens according to the set focus.

The control unit generates control signals for driving and adjusting the variable-focus lens based on the information received from the gaze analysis unit, and collects feedback signals indicating the user's response to the set focus. The collected user response information is then transmitted to the gaze analysis unit and the data learning unit.

The data learning unit 500 collects and stores initial data for training the focus prediction model used to drive the variable-focus lens. When the focus information predicted by the focus prediction model does not match, the data learning unit adds the user response information as new data to the training dataset and manages the newly added information.

In addition, the data learning unit monitors whether errors occur in the focus information predicted by the gaze analysis unit due to vision changes caused by aging or environmental factors. If the frequency of focus prediction errors exceeds a predetermined threshold, the data learning unit retrains the focus prediction model using newly added information—i.e., the user response information—and updates the model used by the gaze analysis unit with the retrained version.

Through this process, the visual assistance device can continuously provide user-customized focus control. Accordingly, the present disclosure improves the accuracy of the focus prediction model by utilizing data from multiple users and enhances the model by reflecting the experience of individual users. Furthermore, the visual assistance device stores changing user data in a cloud or local storage, and continuously updates and trains the focus prediction model (or AI model) using the stored data.

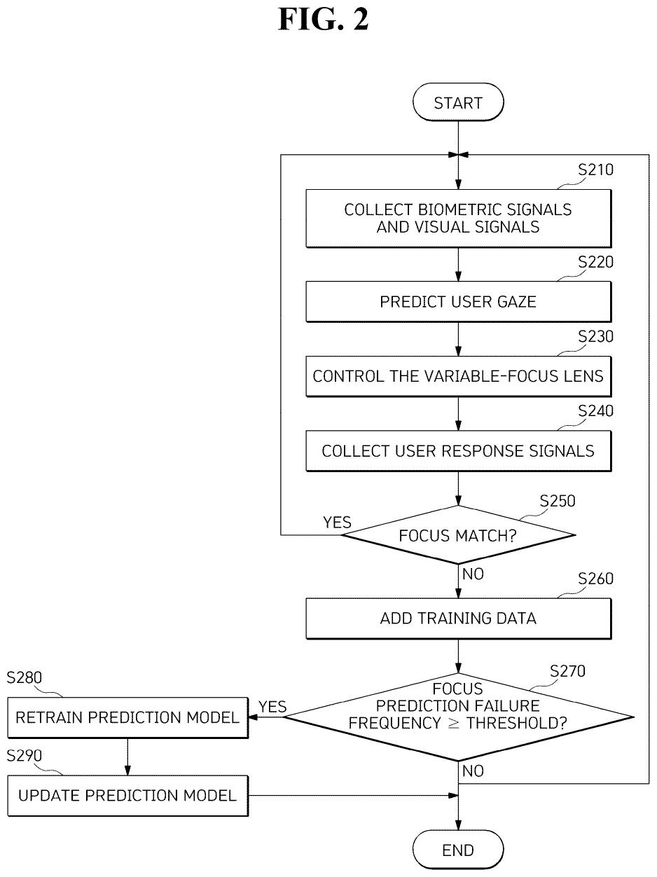

FIG. 2 is a diagram illustrating an example of an operation method of the visual assistance device through progressive learning, as proposed in the present disclosure.

Referring to FIG. 2, the visual assistance device collects the user's biometric signals and visual signals (S210).

Then, the visual assistance device predicts the user's gaze using gaze information (S220). Based on the predicted gaze result, the device controls the variable-focus lens (S230). Subsequently, the visual assistance device collects the user's response signal related to the lens control (S240), and determines whether the focus of the variable-focus lens matches the intended focus based on the collected response signal (S250). If it is determined that the focus does not match, the visual assistance device adds the collected user response signal to the training data and updates the dataset accordingly (S260).

The visual assistance device compares the frequency of focus prediction failures with a predetermined threshold (S270). If the frequency is greater than or equal to the threshold, the device retrains the focus prediction model and updates it accordingly (S280-S290).

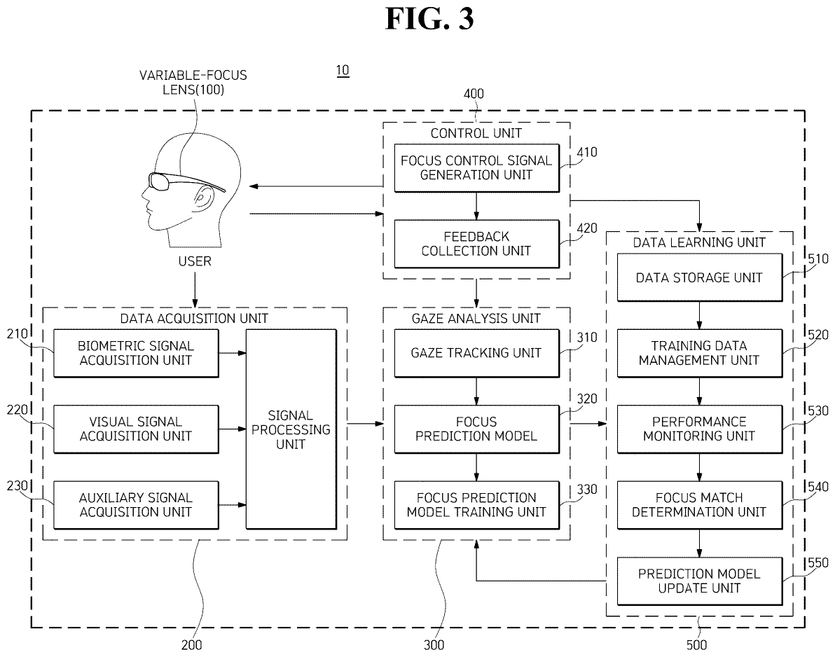

FIG. 3 is a detailed block diagram illustrating an example of the internal configuration of the visual assistance device proposed in the present disclosure.

Referring to FIG. 3, the data acquisition unit 200 may include a biometric signal acquisition unit 210, a visual signal acquisition unit 220, an auxiliary signal acquisition unit 230, and a signal processing unit 240 to collect visual information—i.e., the position, distance, and direction of the object currently being gazed at by the user—from visual input such as images.

The biometric signal acquisition unit 210 performs the following functions:
(1) collects EOG signals by detecting electrical signals around the eyes to precisely track eye movements;
(2) collects EEG signals by detecting brainwaves to track the user's level of concentration, fatigue, and gaze direction; and
(3) collects additional biometric signals such as heart rate and skin conductance to assist in assessing the user's condition.

The visual signal acquisition unit 220 collects distance information to the object being gazed at by utilizing image information or LiDAR sensors required to monitor the user's eye and gaze in order to analyze gaze position and focus.

The auxiliary signal acquisition unit 230 collects supplementary signals using IMU sensors, ambient light sensors, and infrared sensors to obtain information such as movement or brightness level. These signals are used to enhance gaze accuracy and improve the precision of focus alignment under various conditions.

The signal processing unit 240 performs basic signal processing and image processing on the collected biometric signals, visual signals, and auxiliary signals to extract or generate the user's gaze information.

The gaze analysis unit 300 analyzes the user's gaze position, gaze movement path, and fixation time based on the biometric signals (e.g., EOG, EEG) and visual data collected by the data acquisition unit, and performs the function of predicting the object currently being gazed at by the user. The gaze analysis unit provides core data required for the focus prediction model, thereby enabling the control and adjustment of the variable-focus lens.

To achieve this, the gaze analysis unit 300 may include a gaze tracking unit 310, a focus prediction model 320, and a focus match determination unit 330.

The gaze analysis unit tracks the user's gaze movement path in real time through the gaze tracking unit and accurately predicts the object being gazed at, thereby providing necessary data for the focus prediction model. Specifically, the gaze analysis unit calculates the user's gaze movement path using preprocessed EOG and visual data. For example, when the user shifts their gaze from the left to the right side of the screen, the gaze analysis unit analyzes the speed and direction of the gaze movement to track the path. In addition, when the user fixates on a specific object for a certain period of time, the gaze analysis unit determines that the object is being gazed at and records the fixation time. For instance, if the user gazes at the text located at the center of the screen for more than two seconds, the gaze analysis unit recognizes the text as the target of attention and delivers the corresponding data to the focus prediction model. Accordingly, the gaze analysis unit classifies the user's gaze patterns based on gaze movement paths and fixation time on objects or targets, and extracts key features for focus control. For example, the gaze analysis unit may distinguish between scanning patterns and concentration patterns and generate corresponding focus control data for each pattern.

The gaze analysis unit calculates the position and distance of the object being gazed at by the user based on the user's gaze information and inputs this data into the focus prediction model. For example, when the user is looking at a screen located 1.5 meters away, the gaze analysis unit generates the corresponding distance information as an input value for the model. In addition, the gaze analysis unit classifies the type of object being gazed at (e.g., text, image, or video) and the environmental context (e.g., indoor or outdoor), and provides this as additional input to the focus prediction model. For instance, the gaze analysis unit distinguishes between gaze information obtained when the user is viewing a digital screen indoors and when viewing a sign outdoors, and provides that contextualized data to the model.

Additionally, the gaze analysis unit continuously updates the focus prediction model by learning the user's gaze behaviors and adapting to various conditions. To this end, the gaze analysis unit analyzes repeated gaze movement paths and fixation times to learn the user's gaze behavior patterns and updates the focus prediction model based on the learned patterns. For example, if the user tends to move their gaze quickly while viewing specific types of content (e.g., text), the gaze analysis unit may add such patterns to the training data. It may also adapt the focus prediction model based on gaze data collected while the user operates the lens in various environments. For instance, the gaze analysis unit learns the differences in gaze movement patterns between indoor lighting and outdoor lighting and provides environment-optimized focus adjustment accordingly. Furthermore, the gaze analysis unit utilizes user profiling data to train and update (or improve) a user-customized focus prediction model. For example, in the case of a user with presbyopia who frequently gazes at nearby objects, the gaze analysis unit reflects such characteristics in training the focus prediction model.

In a specific example, while the user is reading a document on a computer screen, the gaze analysis unit monitors the user's EOG signals and pupil position data in real time. Each time the user changes lines, the gaze analysis unit analyzes rapid gaze shifts and records the fixation time on each line of text, thereby predicting that the user is reading the text and inputting that information into the focus prediction model. In another scenario, while the user is walking outdoors and gazing at a sign using the variable-focus lens, the gaze analysis unit analyzes visual data and EOG signals to calculate the distance and direction of the sign being viewed. Based on this, the gaze analysis unit generates data for adjusting the focus toward the location of the sign. When the user is watching a video on a smartphone, the gaze analysis unit analyzes the duration for which the user gazes at specific regions of the screen (e.g., subtitles or a specific person). For instance, if the user's gaze is fixed downward while reading subtitles, the gaze analysis unit recognizes this pattern and provides the relevant information to the focus prediction model for appropriate focus control.

The focus match determination unit 330 of the gaze analysis unit performs a key role in determining whether the focus information generated by the focus prediction model matches the user's actual gaze intent. Through this process, the gaze analysis unit evaluates the accuracy of the focus control and, when necessary, generates feedback data for model improvement and retraining. The focus match determination unit monitors various biometric signals (e.g., EOG, EEG) and user response data in real time to determine whether the predicted focus aligns with the user's actual gaze, and provides the corresponding feedback to the data learning unit. In the case of EOG signals used as user response information, experimental studies have shown that when the focus matches, EOG signals remain stable, while mismatched focus may cause variations in EOG signals due to actions such as squinting or pupil dilation. In addition, classification between matched and mismatched EOG signal states may be performed using statistical results derived from analysis of user-collected EOG data during the initial training stage of the focus prediction model. For example, when the gaze analysis unit receives predicted focus information indicating that the user is gazing at an object 3 meters away, the gaze analysis unit collects real-time biometric and response signals—such as EOG, EEG, and heart rate—from the control unit. If the user's eye movement does not correspond to a focus on the object 3 meters away, the system may determine that a mismatch has occurred. Furthermore, the gaze analysis unit may apply a dedicated algorithm to compare the predicted focus position with the actual response data in order to determine focus alignment.

For instance, if the predicted focus position and the actual gaze fall within ±0.5 degrees, the system may determine that a match has occurred; otherwise, it is judged as a mismatch.

The focus match determination unit is capable of accurately determining whether the focus aligns with the user's intended gaze based on various biometric signals such as EOG and EEG. Specifically, the focus match determination unit analyzes the user's EOG signals to calculate gaze movement paths and speeds, and determines whether the predicted focus matches the actual gaze trajectory. For example, when the user gazes at a specific position on a screen, the focus match determination unit tracks voltage fluctuation patterns in the EOG signals to verify whether the predicted position corresponds to the actual gaze. In addition, the focus match determination unit may evaluate the appropriateness of focus adjustment by monitoring changes in the user's concentration level and fatigue after the focus is adjusted. For instance, if the focus is mismatched, the focus match determination unit may detect a decrease in concentration or an increase in fatigue from the EEG signals and use such patterns as indicators of mismatch. Furthermore, the focus match determination unit may also analyze other biometric signals—such as heart rate and skin conductance—in an integrated manner to assess focus accuracy. For example, if the user experiences stress due to an incorrect focus, and the heart rate increases, the system may use this as additional evidence of mismatch.

In a more specific example, when the user is wearing the variable-focus lens while reading a book, the focus match determination unit monitors the user's EOG signals in real time to track the gaze movement path. As the user moves to the next line of text, the focus match determination unit compares the predicted focus transition path with the actual EOG signal patterns to determine whether the focus was accurately adjusted. In another scenario, when the user is engaging in outdoor sports while using the variable-focus lens, the focus match determination unit analyzes rapid gaze shifts in real time. For instance, when the user is tracking a fast-moving ball, the unit determines whether the predicted gaze trajectory generated by the focus prediction model aligns with the user's actual gaze response, and adjusts the focus if necessary. In yet another example, when the user shifts their gaze from a computer monitor to a smartphone, the focus match determination unit monitors EOG and EEG signal changes that occur with transitions between digital screens. The unit determines whether the predicted focus transition has been correctly executed, and if a mismatch is detected, it can immediately generate feedback to be used for improving the focus prediction model.

The control unit 400 may include a focus control signal generation unit 410 and a feedback collection unit 420.

The focus control signal generation unit 410 generates control signals for the variable-focus lens based on the focus prediction information received from the gaze analysis unit, and transmits the generated control signals to the variable-focus lens to adjust the focus in real time.

First, the focus control signal generation unit receives predicted data in real time from the gaze analysis unit regarding the user's gaze position, distance, and direction. If the received prediction data indicates that the user is gazing at a screen located 2 meters away, the focus control signal generation unit analyzes the data to derive the necessary adjustments for controlling the focus of the variable-focus lens. For example, the focus control signal generation unit compares the predicted gaze position with the current focus state of the variable-focus lens to determine whether the lens focus needs to be adjusted from near to far distance. The required adjustments for focus control are converted and optimized into signal forms suitable for electrical or mechanical actuation of the variable-focus lens. For instance, the focus control signal generation unit may convert the prediction data into a voltage or current signal for driving a specific actuator of the variable-focus lens. Subsequently, the focus control signal generation unit issues a command to drive the lens in accordance with the predicted focus position. As an example, if the current focus is set to 1 meter and the predicted focus is 2 meters, the focus control signal generation unit issues a command to adjust the variable-focus lens for long-distance focus.

The focus control signal generation unit generates correction commands to finely adjust the focus of the variable-focus lens when necessary, based on feedback data received after focus adjustment. For example, if the focus is slightly off, the focus control signal generation unit performs minor adjustments to bring the lens into precise focus. Additionally, the focus control signal generation unit may generate multi-stage control signals to smooth the transition of focus and provide the user with a more comfortable visual experience. For instance, during a focus shift, the unit may gradually increase or decrease the control signal to reduce sudden changes, thereby minimizing user discomfort or dizziness caused by abrupt visual transitions.

For example, during computer screen use, when the user shifts their gaze from on-screen text to the keyboard, the focus control signal generation unit receives a focus shift request from the gaze analysis unit—from a distance of 50 cm to 30 cm. In this case, the focus control signal generation unit adjusts the control signal in stages to smoothly transition the focus and minimize visual fatigue for the user. In an outdoor environment, when the user is walking while wearing the variable-focus lens and scanning the surroundings, the focus control signal generation unit adjusts the focus shift signal in real time based on the distance and gaze direction. For instance, if the user gazes at a sign located 2 meters ahead and then shifts their gaze to a road sign 5 meters away, the focus control signal generation unit immediately modifies the signal to adjust the focus accordingly. Additionally, in response to user feedback indicating that the focus shift was too slow in a specific environment, the focus control signal generation unit adjusts the transition speed based on the received feedback. Subsequently, when the user operates the lens in the same environment again, the unit applies a faster transition speed to improve the user's visual satisfaction.

The feedback collection unit performs the function of collecting user response information in real time, synchronized with the timing of focus adjustment of the variable-focus lens, while the user is using the lens at the adjusted focus setting. This collected information is utilized for training and updating the focus prediction model. In particular, the feedback collection unit monitors the user's visual responses and biometric signals (e.g., EOG, EEG) collected through sensors attached to the temple portions of the wearable device in the form of eyeglasses. Based on this monitoring, the unit evaluates the accuracy of the focus prediction and, if necessary, provides feedback data for retraining the focus prediction model.

More specifically, the feedback collection unit collects biometric signals and response information in real time when the user is wearing the variable-focus lens and either gazing at a specific object or experiencing a focus change. It detects EOG signal patterns associated with user discomfort caused by focus mismatch. For example, if the user squints or exhibits repetitive micro-movements of the pupils, the feedback collection unit interprets such behavior as an indication of focus mismatch and collects the corresponding data as feedback. Additionally, the feedback collection unit monitors EEG signals while the user is using the lens to detect changes in concentration or fatigue levels. For instance, if there is a sudden drop in concentration or an increase in signals indicating fatigue, the unit determines that the focus adjustment is inadequate and records the observation as feedback data. When necessary, the feedback collection unit may further collect supplementary biometric signals, such as heart rate and skin conductance, to comprehensively evaluate changes in the user's physiological state. For example, when a sudden change in heart rate or a variation in skin conductance is detected, the feedback collection unit interprets this as an indication of user stress and utilizes it as feedback information for updating the focus prediction model. The collected feedback data is filtered to remove noise or erroneous signals so that only high-reliability data can be used for training. For instance, the feedback collection unit distinguishes between natural eye-blinking behavior and user responses caused by focus mismatch. In addition, the feedback collection unit classifies and labels the collected data according to various environmental conditions so that specific feedback information can be reflected during model training. For example, the feedback collection unit may label focus mismatch data experienced by the user outdoors as “outdoor—bright lighting” and ensure that the model is trained under such labeled conditions. The labeling information generated by the feedback collection unit may be integrated and managed by the training data management module of the data learning unit.

Additionally, the feedback collection unit may provide a user interface for evaluating user satisfaction. In this case, the feedback collection unit offers an interface through which the user can assess their satisfaction or discomfort with the focus adjustment while wearing the lens. For example, the feedback collection unit may provide immediate feedback options through a smartphone application or voice recognition, such as the user stating, “The current focus is incorrect.” Furthermore, when the user experiences focus adjustment issues in a specific situation, the feedback collection unit allows the user to input or record the feedback directly. For instance, if the user is in a new environment with frequent gaze shifts, the feedback collection unit may allow the feedback to be recorded via text or voice input and used for model improvement. These capabilities of the feedback collection unit enable the focus prediction model to be adjusted in real time according to user-provided feedback, thereby allowing immediate adaptation to user needs. For example, if the user provides feedback indicating that the focus adjustment is too slow, the feedback collection unit transmits the information to the prediction model update module of the data learning unit to guide the adjustment of focus transition speed.

For example, when the user is watching TV indoors while using the variable-focus lens, the feedback collection unit monitors EOG signals and changes in concentration in real time. If it detects frequent gaze shifts while the user is reading subtitles on the TV screen, and moments where the focus does not align, the feedback collection unit records such instances as feedback data and utilizes them as training data for improving the model. Additionally, when the user is outdoors and gazing at the surroundings while moving, the feedback collection unit collects supplementary biometric signals such as changes in heart rate along with the EOG signals to form feedback data. If the user experiences a gaze mismatch while focusing on a specific object, the feedback collection unit detects this in real time and reflects it as feedback data for model refinement. If the user feels that the focus adjustment is too slow in a work environment and submits feedback such as “increase focus transition speed” via a smartphone app or voice input, the feedback collection unit receives this information and delivers it to the prediction model update module of the data learning unit in real time, thereby enabling immediate model adjustment.

The data learning unit 500 trains the focus prediction model used for focus estimation. To this end, the data learning unit may include a data storage unit 510, a training data management unit 520, a performance monitoring unit 530, a focus prediction model training unit 540, and a prediction model update unit 550.

The data learning unit tracks the results from the focus match determination unit and adds user response information, delivered as feedback signals, to the training data in order to retrain and update the focus prediction model according to the user's context. The initial focus prediction model required for first-time use of the variable-focus lens is trained using biometric signals, visual signals, and other auxiliary information collected by the data acquisition unit, which are stored in the data storage unit 510. For effective use of the wearable-type variable-focus lens, it is important to minimize the need for additional equipment to extract gaze information from the user. Accordingly, while biometric signals, visual signals, and auxiliary information may be used during the initial model training phase, after this phase is completed, only user response information may be used to ensure convenience in usage. This process may be implemented by performing progressive learning through distinct training phases—namely, the initial phase, adaptation phase, and operational phase—of the focus prediction model.

The training data management unit 520 performs the role of effectively collecting, storing, and managing data necessary for training the focus prediction model, and provides optimal data sets for model training and updating. In particular, the training data management unit analyzes the determination results transmitted from the focus match determination unit, and if a mismatch is detected, it systematically manages and labels various data—including user response information transmitted from the feedback collection unit such as biometric signals (e.g., EOG, EEG), visual signals, and environmental signals—to improve the user-customized focus adjustment model. It also continuously adds and updates new data to enhance the performance of the model.

For example, the training data management unit collects real-time EOG signals and gaze pattern data from the user while the user is working indoors wearing the variable-focus lens, and adds this information to the existing training data. When the user begins working in a new environment (e.g., one that involves frequent monitor viewing), the unit continuously accumulates environment-optimized data for use in model training. Additionally, if the user begins to experience presbyopia, the training data management unit collects new training data that reflects the user's age and changes in vision condition. By collecting gaze patterns and focus adjustment response data specific to presbyopic conditions, the unit supports the updating of existing models or the training of new models so that the user can comfortably use the lens even while experiencing presbyopia. Furthermore, the training data management unit evaluates the validity of data collected over a certain period and deletes or updates data that may negatively impact the performance of the prediction model. For instance, if it determines that data previously collected in an indoor environment is no longer valid after the user transitions to an outdoor setting, the unit removes the outdated data or updates it to match the new environment. To perform these functions, the training data management unit handles data collection and storage, data preprocessing, training data updates and management, and user-specific data management.

Specifically, the training data management unit efficiently collects biometric signals, visual signals, and environmental signals necessary for model training, and securely stores and manages this data. To this end, it collects biometric signals such as the user's EOG and EEG in real time and utilizes them as training data for the focus prediction model. For example, the training data management unit continuously collects EOG signal patterns that appear when the user gazes at objects at specific distances and stores gaze movement data across various conditions. Additionally, the training data management unit may collect environmental information from visual signals, LiDAR, and infrared sensors used for tracking the user's gaze. For instance, it collects and stores data that can analyze gaze movement patterns in low-light environments or focus changes based on distance, and when necessary, ensures that the collected data is securely stored and maintains data integrity and security.

The data collected by the training data management unit may undergo a refinement process to remove noise and irrelevant information in order to improve the efficiency of model training and convert it into a form suitable for learning. In particular, the training data management unit filters out noise or erroneous data that may occur in EOG signals, EEG signals, and the like to ensure the reliability of the data. For example, data containing noise caused by head movement or eye blinking may be removed or corrected. In the case of additionally collected data, the training data management unit normalizes the range and scale of the data to provide consistency in model training. For instance, it may normalize EOG signal voltage values to a fixed range or standardize gaze position data to enhance training efficiency. Finally, the training data management unit extracts key features such as gaze patterns, eye movement speed, and reaction time, and converts them into key parameters that can be used in training the focus prediction model.

To maintain the timeliness and accuracy of the model, the training data management unit manages existing training data and adds new user data. Specifically, it continuously collects real-time user response information and environmental variation data and integrates them with the existing dataset. For example, the training data management unit adds newly collected gaze data to the existing dataset when the user uses the variable-focus lens in new environments such as outdoor activities or low-light conditions. In addition, it evaluates the validity of the collected data and deletes or updates outdated or erroneous data that is no longer suitable for model training. For instance, if gaze data collected after a certain point no longer aligns with the user's response patterns, the training data management unit updates or removes the data to maintain the accuracy of model training.

In addition, the training data management unit manages personalized data based on user profiles to provide an optimized focus prediction model for each individual user. To achieve this, the training data management unit creates and manages profiles that include factors such as the user's age, vision condition, and usage patterns in different environments. For example, the training data management unit separately manages data for users with emerging presbyopia and users with normal vision, labels their response data accordingly, and clarifies response patterns for specific situations or environments to support personalized model training. If a user experiences difficulty adjusting focus in a low-light environment, the unit labels the corresponding EOG signal data and utilizes it for model retraining. Additionally, the training data management unit dynamically provides training data based on the user's real-time responses in order to train a user-customized focus prediction model. Through these functions, the system can implement a model optimized for each user and provide personalized visual focus adjustment functionality.

The performance monitoring unit 530 monitors the results from the focus match determination unit and continuously evaluates the performance of the focus prediction model in accordance with factors such as changes in the user's vision, aging, and environmental conditions. When the accuracy of the model declines, the unit determines that retraining is required. If the predicted focus from the focus prediction model does not match over a certain period or number of occurrences, the performance monitoring unit initiates retraining by instructing the focus prediction model training unit 540 to perform new training. This process enables more efficient synchronization and mapping of biometric signals and response information from the user, thereby enhancing the usability of the variable-focus lens and the accuracy of focus prediction. The operations of the performance monitoring unit may be characterized by performance analysis, performance monitoring, retraining instruction, and model update decision-making.

The performance monitoring unit processes the user's gaze information and feedback data to evaluate the prediction accuracy and overall performance of the focus prediction model. To this end, the performance monitoring unit compares the focus adjustment results of the variable-focus lens worn by the user with the actual gaze data to assess prediction accuracy. For example, when the user is gazing at a specific object, the performance monitoring unit analyzes EOG signals to verify the degree of alignment with the expected gaze pattern. If prediction errors repeatedly occur over a certain period of time, the performance monitoring unit detects performance degradation in the model. For instance, if more than five prediction errors occur within a 10-minute period, the unit considers this a sign of performance decline and uses it as an indicator for retraining. Additionally, the performance monitoring unit analyzes the user's responses—such as EOG and EEG signals, or changes in gaze behavior—to assess the user's level of satisfaction or discomfort related to focus adjustment. For example, if the user squints or blinks rapidly more frequently due to focus misalignment, the unit interprets this as a performance degradation.

The performance monitoring unit monitors changes in the performance of the model due to long-term factors such as aging and changes in vision, and uses the results to determine whether model retraining is necessary. Specifically, the performance monitoring unit utilizes user profiling data to track long-term changes such as age-related decline or vision deterioration. For example, if the user reaches an age where presbyopia typically begins or if changes in myopia or hyperopia are detected through vision test data, the unit may use such conditions as indicators for retraining. In addition, the performance monitoring unit evaluates performance changes according to the user's primary usage environments—such as indoor, outdoor, or low-light settings—by monitoring shifts in usage patterns. For instance, if the user begins using the device more frequently outdoors and prediction errors increase, the unit may detect this change and determine that the model needs to be updated to better fit the new environment. Moreover, by collecting long-term data on individual users, the unit analyzes focus prediction error patterns and identifies whether errors occur frequently during specific times of day or under certain environmental conditions. For example, if prediction errors are often detected while viewing digital screens during the evening, the unit may determine that additional model training is needed for that specific time period.

The performance monitoring unit determines whether the focus prediction model should be retrained based on the analysis results and instructs the prediction model update unit 550 to carry out the update. To achieve this, the performance monitoring unit defines retraining criteria—such as prediction error frequency, user discomfort level, and changes in vision—and triggers the model update once the criteria are met. For example, if the focus prediction error rate exceeds 20% over the course of a month, the unit determines that retraining is necessary. Furthermore, the performance monitoring unit analyzes the user's usage patterns and environment to recommend the optimal timing for model updates. For instance, it may recommend updating the model before the user begins using the lens in a new environment, such as during travel or in a new work setting. When the model is updated, the performance monitoring unit evaluates the initial performance and collects user feedback to verify the precision of the updated model. If necessary, it monitors user satisfaction and prediction accuracy during the first week following the update to determine whether additional improvements are required.

As a more specific example, when the user begins to experience presbyopia due to aging, the existing focus prediction model may no longer provide accurate focus adjustment. The performance monitoring unit detects a trend of increasing prediction errors from the user's EOG signals and focus adjustment patterns and instructs retraining of a new prediction model tailored for presbyopia. In this process, the unit collects the user's vision change data and incorporates it into model training. Additionally, if the user shifts from primarily using the variable-focus lens indoors to frequent outdoor activity, the frequency of focus prediction errors may increase. The performance monitoring unit compares and analyzes biometric signal data between indoor and outdoor environments and determines that the model should be updated to one optimized for outdoor conditions. The new model may be trained to reflect outdoor lighting conditions, distance measurement signals, and other relevant factors to improve focus prediction accuracy. Furthermore, when the user begins using the variable-focus lens with a newly introduced digital device or display environment, the performance monitoring unit may detect that the existing model is not well-suited to that environment. It analyzes changes in gaze patterns and the frequency of focus errors during digital screen viewing and instructs retraining of a model adapted to such conditions. Through this process, the system supports stable focus adjustment for the user even in digital environments.

Next, the present disclosure describes in greater detail the progressive learning method of the focus prediction model, which consists of the initial training phase, the adaptation phase, and the operational phase.

First, the initial training phase is a foundational learning stage in which the manufacturer configures the variable-focus lens and trains a general-purpose focus prediction model. This phase is intended to enable the model to acquire basic focus adjustment capabilities based on generalized user data. In particular, the operation of the initial training phase of the visual assistance device includes general-purpose data collection involving large-scale acquisition of biometric signals (e.g., EOG, EEG, muscle signals around the eyes) and visual signals that are representative of various age groups, vision conditions, and gaze patterns. The visual assistance device then uses the collected data to analyze the correlation between the user's eye movement and the focus of the variable-focus lens in order to train a baseline focus prediction model. For example, when a user gazes at a specific location on a screen, the typical EOG signal patterns observed in such cases are used to train the model, thereby enabling basic gaze prediction functionality.

In addition, the visual assistance device does not incorporate biometric signal changes into the training or updating of the focus prediction model when such signals are generated by user actions or unrelated biometric events that are not associated with the match or mismatch of the set focus. For example, EOG signal variations caused by the user abruptly turning their head or blinking—actions that are not related to focus state—are excluded from focus adjustment. However, when changes in gaze patterns or environmental changes identified through image analysis indicate that such biometric signal variations are relevant to improving the focus prediction model, the system adjusts accordingly to include those data in training. Through this approach, the visual assistance device learns to selectively include or exclude biometric signal variations for model updating, depending on the context in which they occur. Once the baseline model has been trained, the visual assistance device adjusts the model to account for focus changes across generalized visual environments so that the lens can adaptively adjust focus based on varying distances and angles. For example, by analyzing EOG signal patterns from a large number of users gazing at objects located within a specific distance range (e.g., within 1 meter, 1-2 meters, over 2 meters), the system learns the appropriate focus adjustment for each distance. As a result, during the initial training phase, the visual assistance device builds a generalized focus prediction model that can adapt to diverse visual environments and user behaviors.

The adaptive training phase is a process of fine-tuning the model to reflect the individual characteristics of the user. It is typically performed by optical shops or service providers and institutions, where the initially trained model is personalized using actual user data to enable optimized focus prediction based on the user's visual needs and responses. In the adaptive training phase of the visual assistance device, the device generates a user profile by recording factors such as the user's age, vision, and lifestyle habits. For example, in users within the age range where presbyopia typically begins, more training data is incorporated for near-distance focus adjustment. In addition, since the generalized signals used during the initial training phase may cause discomfort for actual users, the adaptive training phase involves additional collection of individual biometric and gaze data. In particular, the model is retrained to reflect the user's unique EOG signal patterns and characteristics such as rapid eye movements. Furthermore, when the predicted focus does not match the user's intent, the system collects feedback based on user responses—such as blinking or gaze shifts—and incorporates it into the model for continuous adjustment. For instance, if the focus set by the model trained in the initial phase does not align with the user's response when gazing at an object 1 meter away, the visual assistance device analyzes specific EOG patterns indicating discomfort and uses this information to perform real-time adjustments to the focus prediction values.

The operational training phase involves training the model to adapt to various visual environments encountered while the user wears the variable-focus lens during daily activities. In particular, this phase maximizes user convenience by relying solely on EOG signals collected from sensors attached to the temple portion of the visual assistance device (e.g., glasses) to train the focus prediction model, while still enabling model retraining through analysis by the focus match determination unit of the gaze analysis unit and the performance monitoring unit of the data learning unit, thereby minimizing the impact on the user's everyday life. In this phase, real-time user response data is reflected in model training, allowing the focus prediction model to adapt to user changes over time. For example, if the user wears the variable-focus lens for a set duration each day while working, the system records gaze movement patterns observed during screen-focused tasks and evolves the model into one optimized for the user's work environment. In another case, if the user frequently shifts gaze between a computer screen and paper documents, the system learns focus transitions optimized for such switching behavior to maintain consistent focus. Specifically, the visual assistance device collects real-time EOG signals during use and reflects user feedback regarding the currently set focus. If repeated signals indicating gaze mismatch are detected, the system learns from these mismatch patterns and performs adjustments. Moreover, by reflecting changes in the user's environment—such as indoor lighting conditions, outdoor settings, or digital screen viewing—the system detects focus prediction errors and collects specific response data for each context to improve the model accordingly. Additionally, if performance degradation or vision changes are detected during long-term use, the performance monitoring unit instructs retraining of the model and incorporates new user data to keep the focus prediction model up to date.

The prediction model update unit 550 performs the function of updating the focus prediction model by transmitting the model trained by the focus prediction model training unit 540 to the gaze analysis unit. During this process, various conditions and prerequisites for model updating are considered to ensure that the focus prediction model for the user's gaze is always maintained in an optimal state. The prediction model update unit provides effective model update functionality through a combination of operations such as data transmission, power management, and evaluation of model suitability. For example, before transmitting the newly trained focus prediction model to the gaze analysis unit, the prediction model update unit performs necessary preparatory tasks to minimize errors during the transmission process. As a first step, the unit checks the power supply status of the variable-focus lens device to confirm whether adequate power is available during the model update. For instance, if the battery level is low, the prediction model update unit may postpone the update or notify the user that charging is required. Additionally, when the visual assistance device transmits the model wirelessly, the unit checks whether sufficient network bandwidth is available. If the Wi-Fi or Bluetooth connection is unstable, the prediction model update unit switches to a transmission standby mode until a stable connection is restored.

In addition, the prediction model update unit monitors in real time the operational status of the existing model currently deployed in the gaze analysis unit to determine whether an update is necessary. The conditions for determining whether a model update is required may include the frequency of focus prediction errors related to the user's gaze, changes in the usage environment, and variations in biometric signal patterns. For example, if frequent prediction errors occur in a particular environment—such as outdoors, indoors, or during computer screen work—the prediction model update unit determines that the model should be updated to one that better suits the given situation. Before transmitting the newly updated model to the gaze analysis unit, the prediction model update unit performs a model suitability verification process to assess whether the new model is appropriate for the current usage environment and user data. For instance, a model suitable for an elderly user may differ from one optimized for a younger user, and the unit selects and updates the appropriate model based on such factors. The updated model, once transmitted, is immediately applied to the gaze analysis unit so that focus control is performed according to the new prediction algorithm.

After the model is applied, an initial performance testing process is performed to verify that the updated model has been properly implemented and is functioning correctly, and to make additional adjustments as needed to maintain optimal performance. In a specific embodiment, immediately after the model is updated, the prediction model update unit monitors the user's gaze data and focus adjustment status for a certain period to evaluate the performance of the new model. For example, during the first 10 to 20 minutes after the update, the system collects data on gaze prediction accuracy and focus adjustment error frequency to determine whether the new model provides improved focus prediction compared to the previous model. To support this evaluation, the prediction model update unit receives user response information as feedback and uses it to assess the real-time performance of the model. For instance, a user interface may be provided to allow the user to rate their satisfaction with the updated model's focus adjustment, and if a substantial amount of negative feedback is collected, a re-update may be considered. If the results of the initial performance test are satisfactory, the model is maintained in a stable state while additional training data is collected to continuously enhance model accuracy. For example, in the case of users who frequently use the system outdoors, environment-specific training data may be additionally collected to further improve model precision.

As an example of the actual application of the prediction model update unit, when a user who primarily uses the variable-focus lens indoors begins to engage in more outdoor activities, the accuracy of the existing indoor focus prediction model may decline. The prediction model update unit detects a pattern of increased prediction errors in outdoor environments and determines that the model should be updated to one optimized for outdoor use. During this process, the unit collects real-time user feedback and biometric signal variations to evaluate the accuracy of the new model and makes further adjustments if necessary. Similarly, when a user's vision gradually changes due to aging, the existing model may also require updating. The prediction model update unit monitors such vision changes and updates the model to a new version that reflects age-related vision deterioration. In such cases, the new model is designed to minimize focus adjustment errors associated with presbyopia, and it is trained using data collected by the user profiling module to enable accurate focus prediction under the changed vision conditions.

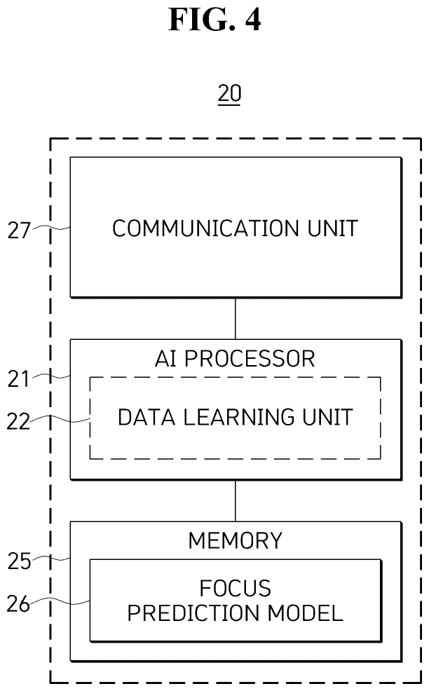

FIG. 4 is a block diagram illustrating an example of an internal configuration of an AI device to which the method proposed in the present disclosure can be applied.

The AI device 20 may include an electronic device incorporating an AI module capable of performing AI processing, or a server including such an AI module. The AI device 20 may also be configured as part of an electronic device to perform at least a portion of AI processing in conjunction with the device.

The AI device 20 may include an AI processor 21, memory 25, and/or a communication unit 27.

As a computing device capable of training neural networks, the AI device 20 may be implemented in various types of electronic devices such as a server, desktop PC, laptop PC, or tablet PC.

The AI processor 21 may train neural networks using programs stored in the memory 25. Examples of neural network models include various deep learning techniques such as deep neural networks (DNN), convolutional neural networks (CNN), recurrent neural networks (RNN), restricted Boltzmann machines (RBM), deep belief networks (DBN), and deep Q-networks (DQN), which may be applied in fields such as computer vision, speech recognition, natural language processing, and audio/signal processing.

Meanwhile, the processor configured to perform the aforementioned functions may be a general-purpose processor (e.g., a CPU) or an AI-dedicated processor (e.g., a GPU) optimized for artificial intelligence training.

The AI processor may analyze gaze information related to the user's gaze using visual information of an object, generate focus control information for adjusting the focus of the visual assistance device through the trained focus prediction model, generate a control signal for adjusting the focus of the visual assistance device based on the focus control information, receive user response information regarding the set focus of the visual assistance device as feedback, acquire training data for training the focus prediction model, monitor whether errors occur in the focus control information, and update the focus prediction model by retraining it using the updated training data.

The memory 25 may store various programs and data necessary for the operation of the AI device 20. The memory 25 may be implemented as non-volatile memory, volatile memory, flash memory, a hard disk drive (HDD), or a solid-state drive (SSD). The memory 25 is accessed by the AI processor 21, and the AI processor 21 may perform operations such as reading, writing, modifying, deleting, or updating data. Additionally, the memory 25 may store a neural network model (e.g., a focus prediction model 26) generated through a training algorithm for data classification or recognition according to one embodiment of the present invention.

Meanwhile, the AI processor 21 may include a data learning unit 22 that trains neural networks for data classification or recognition.

The data learning unit 22 may be implemented in the form of at least one hardware chip mounted in the AI device 20. For example, the data learning unit 22 may be fabricated as a dedicated hardware chip for artificial intelligence (AI), or it may be configured as part of a general-purpose processor (CPU) or a graphics processing unit (GPU) and mounted in the AI device 20. Additionally, the data learning unit 22 may be implemented as a software module. When implemented as a software module (or a program module including instructions), the software module may be stored in a non-transitory computer-readable recording medium. In this case, the software module may be provided by at least one of an operating system (OS) or an application.

The communication unit 27 may transmit AI processing results generated by the AI processor 21 to an external electronic device, and may also receive information from an external electronic device.

Here, the external electronic device may refer to a device capable of communicating with the AI device via wired or wireless communication. The communication unit 27 may receive visual information of an object being gazed at by the user, based on the user's biometric signals, visual signals, and auxiliary signals.

Meanwhile, although FIG. 4 describes the AI device 20 as being functionally divided into components such as the AI processor 21, memory 25, and communication unit 27, it is to be understood that these components may be integrated into a single module referred to as an AI module.

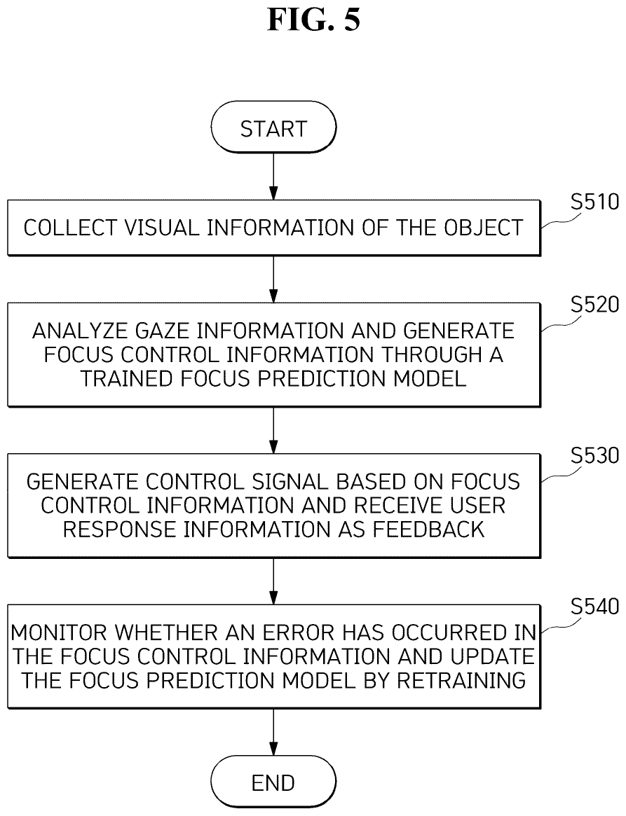

FIG. 5 is a flowchart illustrating an example of a user-customized focus control method through progressive learning as proposed in the present disclosure.

Referring to FIG. 5, the visual assistance device (or variable-focus lens) collects visual information of the object being gazed at by the user based on the user's biometric signals, visual signals, and auxiliary signals (S510).

The biometric signals may include EOG signals, EEG signals, heart rate, and skin conductance; the visual signals may include image information for monitoring the user's eyes and gaze, as well as distance information between the user and the object; and the auxiliary signals may include movement information and brightness information measured by a plurality of sensors.

The visual information of the object may include the position, distance, and direction of the object currently being gazed at by the user within the captured image.

The visual assistance device analyzes gaze information related to the user's gaze using the visual information of the object and generates focus control information to adjust the focus of the visual assistance device through a trained focus prediction model (S520).

In step S520, i.e., the step of generating the focus control information, the device may classify the user's gaze patterns based on the gaze movement path and fixation time information, and generate focus control information corresponding to each gaze pattern. Additionally, the device may further determine whether the set focus matches the user's intended focus based on the response information.

If the set focus does not match, the visual assistance device adds the response information to the training data and updates the training dataset accordingly.

The gaze information may include information about the user's gaze position, information about the movement path of the gaze, and fixation time information for the object being gazed at.

In addition, the visual assistance device generates a control signal to control the focus of the visual assistance device based on the focus control information and receives response information as feedback regarding the user's reaction to the set focus of the visual assistance device (S530).

In addition, the visual assistance device monitors whether errors have occurred in the focus control information and retrains the focus prediction model using the updated training data by updating the training dataset accordingly, thereby updating the focus prediction model (S540).

In step S540, monitoring for errors in the focus control information may be performed by comparing the frequency of focus prediction failures with a predefined threshold.

If the focus prediction failure frequency is greater than or equal to the threshold, the visual assistance device may retrain the focus prediction model.

The embodiments described above are merely examples in which the components and features of the present invention are combined in a predefined manner. Unless explicitly stated otherwise, each component or feature should be considered optional. Each component or feature may be implemented independently of the others. In addition, it is possible to construct embodiments of the invention by combining some of the components and/or features. The order of operations described in the embodiments of the present invention may be modified. A portion of the components or features of one embodiment may be included in another embodiment, or may be substituted with corresponding components or features of another embodiment. It is also evident that claims without explicit citation relationships may be combined to form embodiments, or may be included as new claims through amendments made after filing.

The embodiments according to the present invention may be implemented in various ways, including hardware, firmware, software, or a combination thereof. In the case of hardware implementation, an embodiment of the present invention may be realized using one or more application-specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, or the like.

In the case of firmware or software implementation, an embodiment of the present invention may be realized in the form of modules, procedures, functions, or the like that perform the functions or operations described above. Software code may be stored in memory and executed by a processor. The memory may be located inside or outside the processor and may exchange data with the processor using various known means.

The embodiments according to the present invention may be implemented in various ways, including hardware, firmware, software, or a combination thereof. In the case of hardware implementation, an embodiment of the present invention may be realized using one or more application-specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, or the like.

In the case of firmware or software implementation, an embodiment of the present invention may be realized in the form of modules, procedures, functions, or the like that perform the functions or operations described above. Software code may be stored in memory and executed by a processor. The memory may be located inside or outside the processor and may exchange data with the processor using various known means.

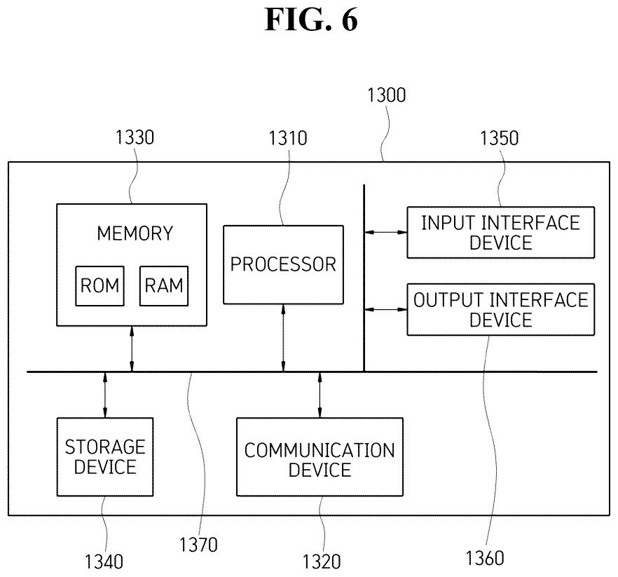

FIG. 6 is a block diagram illustrating a computer system for implementing a method according to an example embodiment of the present disclosure.

Referring to FIG. 6, the computer system 1300 may include at least one of a processor 1310, a memory 1330, an input interface device 1350, an output interface device 1360, and a storage device 1340 communicating with one another through a bus 1370. The computer system 1300 may also include a communication device 1320 coupled to a network. The processor 1310 may be or include a central processing unit (CPU) or a semiconductor device that executes instructions stored in the memory 1330 or in the storage device 1340. The memory 1330 and the storage device 1340 may include various types of volatile or nonvolatile storage media. For example, the memory may include a read-only memory (ROM) and a random access memory (RAM). In example embodiments of the present disclosure, the memory may be located inside or outside the processor, and may be connected to the processor through various known means. The memory is or includes various types of volatile or nonvolatile storage media, and for example, may include a read-only memory (ROM) or a random access memory (RAM).

Accordingly, example embodiments of the present disclosure may be implemented as a method implemented in a computer or a non-transitory computer-readable medium storing computer-executable instructions. In an example embodiment, when executed by the processor, computer-readable instructions may perform a method according to at least one aspect of the present disclosure.

The communication device 1320 may transmit or receive wired signals or wireless signals.

Additionally, the method according to an example embodiment of the present disclosure may be implemented in the form of program instructions that can be executed through various computer means and recorded on a computer-readable medium.

The computer-readable medium may include program instructions, data files, data structures, etc., singly or in combination. The program instructions recorded on the computer-readable medium may be specially designed and configured for the example embodiments of the present disclosure, or may be known and usable by those skilled in the art of computer software. Computer-readable recording media may include a hardware device configured to store and perform program instructions. For example, the computer-readable recording media may be or include magnetic media such as hard disks, floppy disks, and magnetic tapes, optical media such as CD-ROMs and DVDs, magneto-optical media such as floptical disks, ROM, RAM, flash memory, etc. The program instructions may include not only machine language codes such as that generated by a compiler, but also high-level language codes that can be executed by a computer through an interpreter, etc.

What is claimed is:

1. A visual assistance device configured to control focus through progressive learning, comprising:
a data acquisition unit configured to collect visual information of an object being gazed at by a user, based on the user's biometric signals, visual signals, and auxiliary signals;
a gaze analysis unit configured to analyze gaze information related to the user using the visual information of the object, and to generate focus control information for adjusting the focus of the visual assistance device through a trained focus prediction model;
a control unit configured to generate a control signal for controlling the focus of the visual assistance device based on the focus control information, and to receive user response information as feedback regarding the focus set in the visual assistance device; and
a data learning unit configured to acquire training data for training the focus prediction model, to monitor whether errors occur in the focus control information, and to update the focus prediction model by retraining the model using the updated training data.

2. The visual assistance device of claim 1,
wherein the visual information of the object includes information regarding a position, a distance, and a direction of the object currently being gazed at by the user within an image.

3. The visual assistance device of claim 1,
wherein the gaze information includes information regarding a gaze position of the user, a movement path of the gaze, and a fixation time during which the object is gazed at.

4. The visual assistance device of claim 3,
wherein the gaze analysis unit is configured to classify a gaze pattern of the user based on the movement path and the fixation time, and to generate focus control information corresponding to each gaze pattern.

5. The visual assistance device of claim 4,
wherein the gaze analysis unit is configured to determine whether the set focus matches the user's gaze based on the response information.

6. The visual assistance device of claim 5,
wherein, when the set focus does not match, the data learning unit is configured to add the response information to the training data and update the training dataset.

7. The visual assistance device of claim 6,
wherein the data learning unit is configured to monitor whether an error has occurred in the focus control information by comparing the focus prediction failure frequency with a threshold value.

8. The visual assistance device of claim 7,
wherein, when the focus prediction failure frequency is greater than or equal to the threshold value, the data learning unit is configured to retrain the focus prediction model.

9. The visual assistance device of claim 1,
wherein the data learning unit is configured to train the focus prediction model through an initial training phase, an adaptive training phase, and an operational training phase.

10. The visual assistance device of claim 1,
wherein the biometric signals include EOG signals, EEG signals, heart rate, and skin conductance,
wherein the visual signals include image information for monitoring the user's eyes and gaze, and distance information between the user and the object, and
wherein the auxiliary signals include movement information and brightness information measured by a plurality of sensors.

11. A method for controlling focus through progressive learning, comprising:
collecting visual information of an object being gazed at by a user, based on the user's biometric signals, visual signals, and auxiliary signals;
generating focus control information for adjusting the focus of a visual assistance device through a trained focus prediction model, by analyzing gaze information related to the user using the visual information of the object;
generating a control signal for controlling the focus of the visual assistance device based on the focus control information, and receiving user response information as feedback regarding the focus set in the visual assistance device; and
updating the focus prediction model by retraining the model using updated training data, through monitoring whether errors have occurred in the focus control information and updating the acquired training data.

12. The method of claim 11,
wherein the visual information of the object includes information regarding a position, a distance, and a direction of the object currently being gazed at by the user within an image.

13. The method of claim 11,
wherein the gaze information includes information regarding a gaze position of the user, a movement path of the gaze, and a fixation time during which the object is gazed at.

14. The method of claim 13,
wherein the step of generating the focus control information comprises:
classifying a gaze pattern of the user based on the movement path of the gaze and the fixation time; and
generating focus control information corresponding to each gaze pattern.

15. The method of claim 14,
wherein the step of generating the focus control information includes determining whether the set focus matches the user's intent based on the response information.

16. The method of claim 15, further comprising
adding the response information to the training data and updating the training dataset when the set focus does not match.

17. The method of claim 16,
wherein the step of monitoring whether an error has occurred in the focus control information comprises comparing the focus prediction failure frequency with a threshold.

18. The method of claim 17,
wherein, when the focus prediction failure frequency is greater than or equal to the threshold, the focus prediction model is retrained.

19. The method of claim 11,
wherein the biometric signals include EOG signals, EEG signals, heart rate, and skin conductance,
wherein the visual signals include image information for monitoring the user's eyes and gaze, and distance information between the user and the object, and
wherein the auxiliary signals include movement information and brightness information measured by a plurality of sensors.

20. An AI device configured to control focus through progressive learning, comprising:
a communication unit configured to receive visual information of an object being gazed at by a user based on the user's biometric signals, visual signals, and auxiliary signals;
a memory configured to store a focus prediction model; and
an AI processor functionally connected to the communication unit and the memory and configured to control overall operations of the AI device,
wherein the AI processor is configured to analyze gaze information related to the user using the visual information of the object, generate focus control information for adjusting a focus of a visual assistance device through a trained focus prediction model, generate a control signal for controlling the focus of the visual assistance device based on the focus control information, receive response information from the user as feedback regarding a focus set in the visual assistance device, acquire training data for training the focus prediction model, monitor whether errors occur in the focus control information, and update the focus prediction model by retraining the model using the updated training data.
